(19) United States
(12) Patent Application Publication
(10) Pub. No.: US 2015/0012006 A1
(43) Pub. Date: Jan. 8, 2015

(54) ANASTOMOSIS STAPLING/CLIPPING DEVICES, SYSTEMS AND METHODS

(71) Applicant: Cardica, Inc., Redwood City, CA (US)

(72) Inventors: Bernard A. Hausen, Redwood City, CA (US); Bryan D. Knodel, Flagstaff, AZ (US); Stefanos Demertzis, Lugano (CH); Brendan M. Donohoe, Fairfax, CA (US)

(21) Appl. No.: 14/321,757

(22) Filed: Jul. 1, 2014

Related U.S. Application Data
(60) Provisional application No. 61/842,163, filed on Jul. 2, 2013.
(60) Provisional application No. 61/856,638, filed on Jul. 19, 2013.

(51) Int. Cl. A61B 17/115; A61B 17/11; A61B 17/064

(52) U.S. Cl. CPC A61B 17/115; A61B 17/0644; A61B 17/1114; A61B 2017/1107; A61B 2017/1157
USPC 606/108; 606/151

(57) ABSTRACT

An apparatus for performing anastomosis procedures comprises of an end-effector to engage a lumen for attaching a tubular structure to said lumen to create a connective branch or an anastomosis. The apparatus includes a contractible and expandable member coupled to the end-effector to secure engagement of the end-effector with the lumen. A pair of wedge members operates to deploy a staple to attach the tubular structure to the lumen. The pair of wedge members operates in opposing directions to deploy the staple.

### CROSS-REFERENCE TO RELATED APPLICATION

This application claims benefit of and is a non-provisional application of U.S. Provisional Application No. 61/842,163 (Attorney Docket No. 383-1), filed on Jul. 2, 2013, and U.S. Provisional Application No. 61/856,638 (Attorney Docket No. 383-2), filed on Jul. 19, 2013, which are incorporated herein by reference in their entireties.

### FIELD OF THE INVENTION

The present invention relates generally to medical devices, and more particularly to surgical stapling or clip applying systems for attaching or connecting tubular structures (e.g., blood vessels, segments of intestines, prostheses, etc.) in anastomosis procedures.

### BACKGROUND

An anastomosis procedure is a surgical procedure where two separate tubular or hollow structures are surgically grafted together to form a continuous fluid connection, vascular branch or joint between them to facilitate or restore fluid flow. Anastomosis procedures may be used to create bypasses to occluded vessels or passages (e.g., arteries, veins, intestines, etc.), so as to restore flows to and/or from organs in the bodies of patients. Devices that can perform or assist with such important surgical operations are highly desired to reduce the burden and/or time required for performing such complex anastomosis surgical procedures.

### SUMMARY OF THE INVENTION

An apparatus for performing anastomosis procedures comprises of an end-effector to engage a lumen for attaching a tubular structure to said lumen to create a connective branch or an anastomosis. The apparatus includes a contractible and expandable member coupled to the end-effector to secure engagement of the end-effector with the lumen. A pair of wedge members operates to deploy a staple to attach the tubular structure to the lumen. The pair of wedge members operates in opposing directions to deploy the staple or the wedge members operate in a scissor-like motion to deploy the staple. During deployment, the contractible and expendable member provides support to the lumen as the staple is deployed to attach the tubular structure to the lumen. The contractible and expandable member is placed in a contracted state when the end-effector is inserted through the lumen for engagement. The contractible and expandable member is placed in an expanded state to secure the engagement of the end-effector with the lumen. The end-effector can be operated telescopically by extending from a first length to a second length, retracting from the second length to the first length, or varying at a length between the first length and the second length. The end-effector may be substantially rigid, substantially flexible, or substantially steerable. The wedge members may deploy the staple by urging the legs members of the staple in respective arcuate motions for staple deployment.

A staple for performing anastomosis procedures includes a base member with a hinge element near each of ends of the base member, wherein the hinges are oriented substantially on laterally opposite sides of the base member. A leg member extends from each of the hinge elements, wherein each leg member includes a first tine and a second tine. During deployment, each leg member pivots or rotates respectively about the hinge member in an arcuate motion. In a deployed state, the leg member of one end of the base member overlaps or crosses with another leg member of another end of the base member. The hinge elements are non-collinear with the axis of the base member. Each of the hinge elements becomes twisted after the staple is deployed due to the respective rotation of the leg members.

### BRIEF DESCRIPTION OF THE DRAWINGS

The present invention will be readily understood by the following detailed description, taken in conjunction with accompanying drawings, illustrating by way of examples of the invention. The figures are merely exemplary and not limiting. The objects and elements in the drawings are not necessarily drawn to scale, proportion, precise orientation or positional relationships; instead, emphasis is focused on illustrating the principles of the invention. Descriptive terms such as “upper,” “lower,” “upward,” “downward”, “forward”, “backward”, and the like are intended for the convenience of the reader and refer to the orientation and/or motion of parts as illustrated and described; they do not necessarily limit the orientation or operation of the features, aspects, or embodiments of the invention. The drawings illustrate the design and utility of various features, aspects, or embodiments of the present invention, in which like element are typically referred to by like reference symbols or numerals. The drawings, however, depict the features, aspects, or embodiments of the invention, and should not be taken as limiting in their scope. With this understanding, the features, aspects, or embodiments of the invention will be described and explained with specificity and details through the use of the accompanying drawings in which:

FIG. 1A through FIG. 1C illustrate an example of a surgical stapling/clip applying device with specially configured staples/clips for attaching a tubular structure (e.g., harvested vessel, prosthesis, etc.) to a lumen (e.g., artery, vein, intestine, etc.) in an anastomosis surgical procedure, in accordance with features, aspects, or embodiments of the present invention.

FIG. 2A through FIG. 2C illustrate activation of the surgical stapling/clip applying device to allow insertion of an effector into a target lumen to begin the attachment or stapling/clip applying process of a tubular structure to the target lumen, in accordance with features, aspects, or embodiments of the present invention.

FIG. 3A through FIG. 3D illustrate the use of a guide wire to position the surgical stapling/clip applying device for insertion into a target lumen for attaching, stapling, or clipping a tubular structure to the target lumen, in accordance with features, aspects, or embodiments of the present invention.

FIG. 4 illustrate a close-up view of an expandable and contractible support member of the surgical stapling/clip applying device, in accordance with features, aspects, or embodiments of the present invention.

FIG. 5A and FIG. 5B illustrate close-up views of an expandable and contractible support member of the surgical stapling/clip applying device, in accordance with features, aspects, or embodiments of the present invention.

FIG. 6A and FIG. 6B illustrate the end-effector of the surgical stapling/clip applying device engaging with a target lumen, in accordance with features, aspects, or embodiments of the present invention.

FIG. 7A and FIG. 7B illustrate the operation of the surgical stapling/clip applying device in applying staples/clips to connect or attach a tubular structure to a target lumen, in accordance with features, aspects, or embodiments of the present invention.

FIG. 8A and FIG. 8B illustrate the surgical stapling/clip applying device positioned to deploy specially configured staples/clips to attach or connect a tubular structure to a target lumen, in accordance with features, aspects, or embodiments of the present invention.

FIG. 9A illustrates one example of a tubular structure (e.g., harvested vessel, prosthesis, etc.), in accordance with features, aspects, or embodiments of the present invention.

FIG. 9B illustrates a tubular structure (e.g., harvested vessel, prosthesis, etc.) with specially configured staples/clips of the surgical stapling device, in accordance with features, aspects, or embodiments of the present invention.

FIG. 10A illustrates an isometric or perspective view of a specially configured staple/clip of the surgical stapling/clip applying device in its undeployed configuration, in accordance with features, aspects, or embodiments of the present invention.

FIG. 10B illustrates a side view of a specially configured staple/clip of the surgical stapling/clip applying device in its undeployed configuration, in accordance with features, aspects, or embodiments of the present invention.

FIG. 10C illustrates a top view of a specially configured staple/clip of the surgical stapling/clip applying device in its undeployed configuration, in accordance with features, aspects, or embodiments of the present invention.

FIG. 11A illustrates an isometric or perspective view of a specially configured staple/clip of the surgical stapling/clip applying device in its deployed configuration, in accordance with features, aspects, or embodiments of the present invention.

FIG. 11B illustrates a side view of a specially configured staple/clip of the surgical stapling/clip applying device in its deployed configuration, in accordance with features, aspects, or embodiments of the present invention.

FIG. 11C illustrates a top view of a specially configured staple/clip of the surgical stapling/clip applying device in its deployed configuration, in accordance with features, aspects, or embodiments of the present invention.

FIG. 12A illustrates a close-up view of the tubular structure with deployed staples/clips, in accordance with features, aspects, or embodiments of the present invention.

FIG. 12B illustrates, for greater clarity, a further close-up view of the tubular structure with a deployed staple/clip, in accordance with features, aspects, or embodiments of the present invention.

FIG. 13A illustrates a tubular structure to be attached or connected to a target lumen with an undeployed staple/clip, in accordance with features, aspects, or embodiments of the present invention.

FIG. 13B illustrates a tubular structure attached or connected to a target lumen with a deployed staple/clip, in accordance with features, aspects, or embodiments of the present invention.

FIG. 14A illustrates the staple/clip deployment mechanisms of the surgical stapling/clip applying device, in accordance with features, aspects, or embodiments of the present invention.

FIG. 14B illustrates a close-up view of the staple/clip deployment mechanisms of the surgical stapling/clip applying device, in accordance with features, aspects, or embodiments of the present invention.

FIG. 15A illustrates, for clarity, one leg/tine of the staple/clip being deployed by the staple/clip deployment mechanisms of the surgical stapling/clip applying device, in accordance with features, aspects, or embodiments of the present invention.

FIG. 15B illustrates, for clarity, a close-up view of one leg/tine of the staple/clip being deployed by the staple/clip deployment mechanisms of the surgical stapling/clip applying device, in accordance with features, in accordance with features, aspects, or embodiments of the present invention.

FIG. 15C illustrates the staple/clip being deployed by the staple/clip deployment mechanisms of the surgical stapling/clip applying device, in accordance with features, aspects, or embodiments of the present invention.

FIG. 15D illustrates a slightly zoomed-out view of the end-effector to show details of the staple/clip deployment mechanisms, in accordance with features, aspects, or embodiments of the present invention.

FIG. 16A through FIG. 16C illustrate the staple/clip deployment mechanisms (sequentially) deploying the staples/clips, in accordance with features, aspects, or embodiments of the present invention.

FIG. 16D illustrates staples deployed to attach or connect a tubular structure to a target lumen, in accordance with features, aspects, or embodiments of the present invention.

FIG. 17A and FIG. 17B illustrate the surgical stapling/clip applying device attaching or connecting a tubular structure to a target lumen, in accordance with features, aspects, or embodiments of the present invention.

FIG. 18A illustrates a cross-sectional view of the staple/clip applying inner support members/structures of the surgical stapling/clip applying device, in accordance with features, aspects, or embodiments of the present invention.

FIG. 18B illustrates a stapling/clip support member/structure of the surgical stapling/clip applying device, in accordance with features, aspects, or embodiments of the present invention.

FIG. 18C illustrates a distal staple/clip retaining, constraining, or supporting member of the surgical stapling/clip applying device, in accordance with features, aspects, or embodiments of the present invention.

FIG. 18D illustrates a cross-sectional view of the staple/clip outer support members/structures of the surgical stapling/clip applying device, in accordance with features, aspects, or embodiments of the present invention.

FIG. 18E illustrates a cross-sectional view of the distal portion of the surgical stapling/clip applying device illustrating various structures and members of the device located at the distal end of the device, in accordance with features, aspects, or embodiments of the present invention.

As can be appropriated, the use of same or similar symbols or numerals in different figures indicates similar, equivalent, or identical items or features.

### DETAILED DESCRIPTION

In the following detailed description, specific details are set forth in order to provide a thorough understanding of the present invention. However, it will be readily understood by those skilled in the art that the present invention may be practiced without these specific details. Alternatively, some of the well-known parts, components, hardware, methods of operations, and procedures may not be described in detail or elaborated so as to avoid obscuring the present invention; but, nevertheless, they are within the spirit and scope of the present invention.

As mentioned, an anastomosis procedure is a surgical procedure where two separate tubular or hollow structures are surgically grafted together to form a continuous fluid connection, vascular branch or joint between them to facilitate or restore fluid flow in various passages, capillaries, or vessels in a patient. Devices that can perform or assist with such surgical operations are highly desired to reduce the burden and/or time required for performing anastomosis surgical procedures. For example, a vascular anastomosis procedure involves creating an anastomosis (e.g., a junction, bypass, or the like) between blood vessels to create or restore blood flow. An anastomosis surgical operation may be necessary when a patient suffers from coronary artery disease (CAD). In such a condition, an occlusion or stenosis in a coronary artery restricts blood flow to the heart muscle. In order to treat CAD, the area where the occlusion occurs is bypassed to reroute blood flow by grafting a vessel, such as a harvested artery or vein, or, in some situation, a prosthesis to provide an alternative route or passage. An anastomosis procedure is performed between a graft vessel or prosthesis and two target vessels in order to bypass the blocked coronary artery. In such a scenario, the bypass circumvents the occlusion and restores adequate blood flow to the heart muscle to prevent cardiac malfunctions. This treatment is commonly known as a coronary artery bypass grafting procedure (CABG). Accordingly, devices that can perform or assist with such important surgical operations are highly desired to reduce the burden and/or time required for performing such anastomosis surgical procedures.

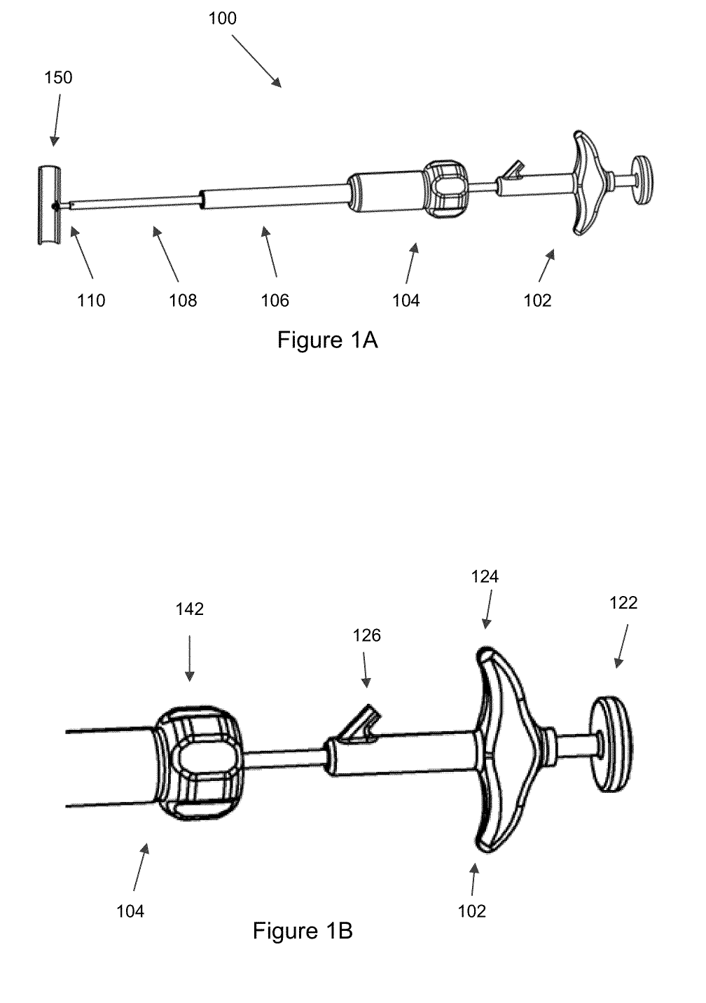

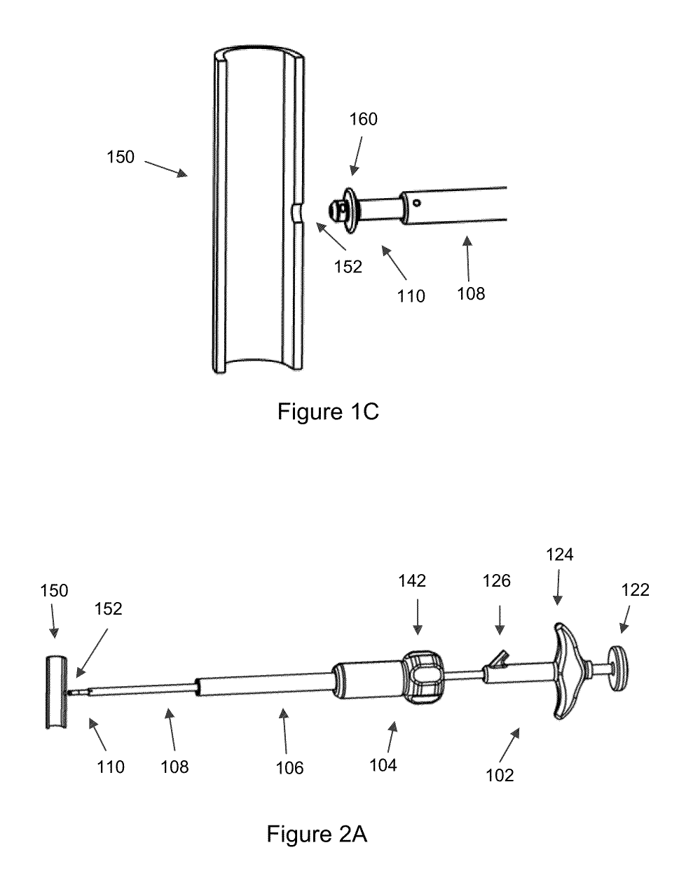

By way of example, FIGS. 1A through 1C illustrate an anastomosis stapling/clip applying device 100 for attaching a tubular structure (e.g., a harvested vessel, a prosthesis, etc.) to a lumen 150 (e.g., a blood vessel, a segment of an intestine, etc.) to create a connection for a bypass to facilitate or restore fluid flow. As illustrated in FIG. 1A, the anastomosis stapling/clip applying device 100 is generally an elongated and tapered instrument comprising of a placement control handle assembly 102, a deployment control handle assembly 104, a deployment shaft assembly 106, a placement shaft member 108, and an application shaft member 110. The anastomosis stapling/clip applying device 100 is designed and constructed to engage with a lumen 150. As such, the substantially elongated and tapered construction is optimized to facilitate maneuverability in substantially small and tight spaces or volumes and to engage with substantially small target lumens. Some sections or portions of the anastomosis stapling/clip applying device 100 may be flexible and/or steerable. As illustrated in FIG. 1B, the placement control handle assembly 102 may include a control plunger member or control articulation member 122, which may be used to articulate the application shaft member 110 in various manners of articulation (e.g., extending and/or contracting the distal portion of the application shaft member 110, twist or turn the distal portion of the application shaft member 110, steer the distal portion of the application shaft member 110, etc.). The handle assembly 102 may also include a handle support member 124 to assist with the control and placement of the stapling device 100 and the operation of the various control and placement mechanisms of the stapling device 100. The handle assembly 102 may further include a through port 126 that provides a pathway from the proximal portion of the stapling device 100 all the way through to the distal tip of the application shaft member 110. For example, in some applications, a guidewire member may be inserted into the through port 126 and maneuvered or threaded through the pathway or passage of the through port 126 all the way to the distal tip of the application shaft member 110 to assist with placement of the application shaft member 110 to engage a target lumen 150 for an anastomosis surgical procedure.

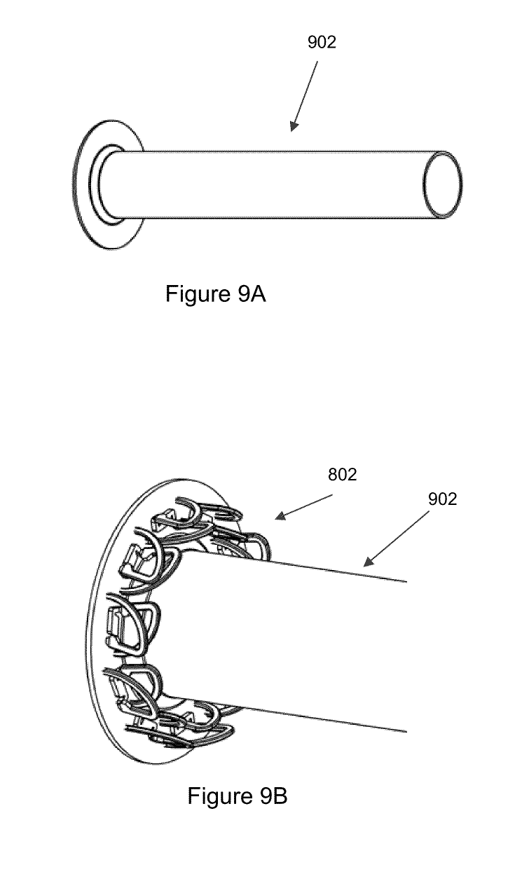

Also illustrated in FIG. 1B, the deployment control handle assembly 104 may include a deployment control knob member 142. To be discussed in further detail, the deployment control knob member 142 may be operated to deploy staples or clips to attach or connect a tubular structure (e.g., a harvested vessel, a prosthesis, etc.) to a lumen 150 (e.g., a blood vessel, a segment of an intestine, etc.) to create a connection or branch for a bypass to facilitate or restore fluid flow. Referring to FIG. 1C, the application shaft member 110 may be slidably coupled to placement shaft member 108. Both of these members 108 and 110 may be used to engage the stapling device 100 to the target lumen 150. Further illustrated in FIG. 1C, the application shaft member 110 includes an expandable and contractible support member 160. As will be discussed in further detail, once the application shaft member 110 has been inserted into the target lumen 150, (initially with the support member 160 in a contracted configuration) the support member 160 can be made to expand into substantially a disc-like support structure or configuration, which can hold the application shaft member 110 in place and properly engaged with the lumen 150. Also, in the expanded configuration, the support member 160 provides certain amount of stiffness and support to an opening 152 of the lumen 150 to assist with the attachment of a tubular structure 902 (see FIG. 9A and FIG. 9B) to the lumen 150 as staples or clips are applied by the stapling device 100.

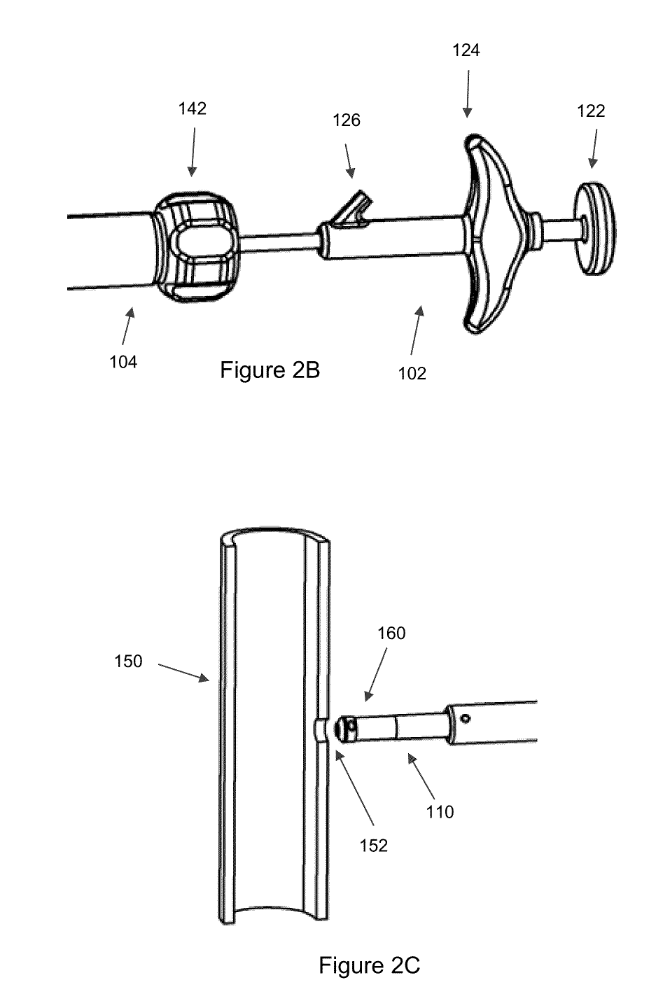

FIGS. 2A through 2C illustrate activation of the control plunger member or control articulation member 122 (e.g., pushing down the control plunger member 122) to extend a portion of the application shaft member 110. As such, the expandable and contractible support member 160 is placed into a contracted configuration, and the distal or tip portion of the application shaft member 110 may be correspondingly placed in its minimum diameter configuration to facilitate insertion of the distal or tip portion of the application shaft member 110 through the opening 152 of the lumen 150. As illustrated in FIG. 2A and FIG. 2B, the control plunger member 122 is pushed “down” (e.g., pushed closer to the handle support member 124), and the tip portion of the application shaft member 110 is extended with the support member 160 contracted, as illustrated in FIG. 2A and FIG. 2C.

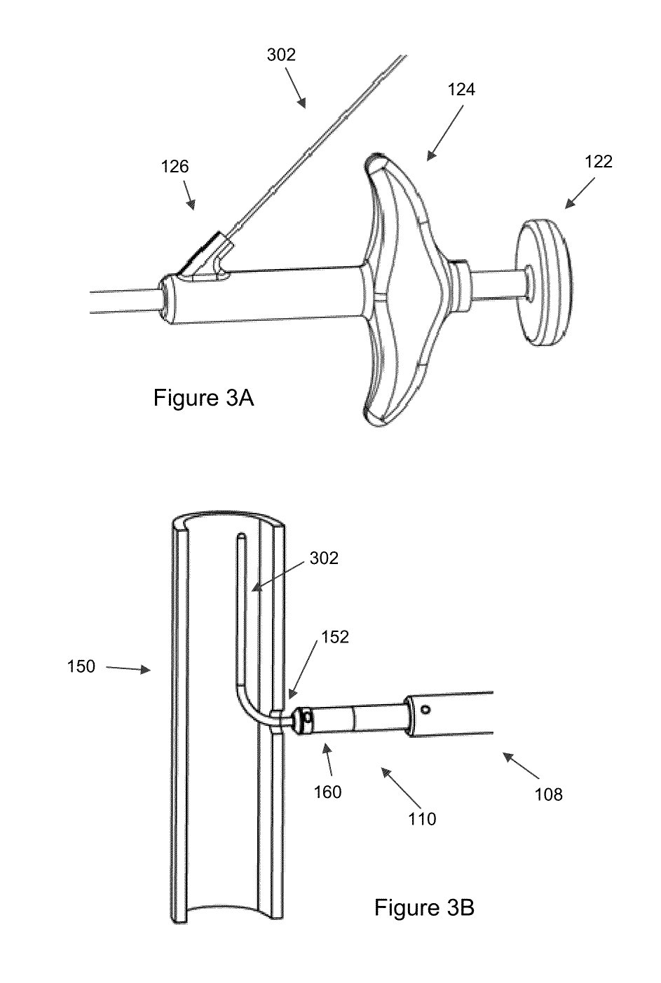

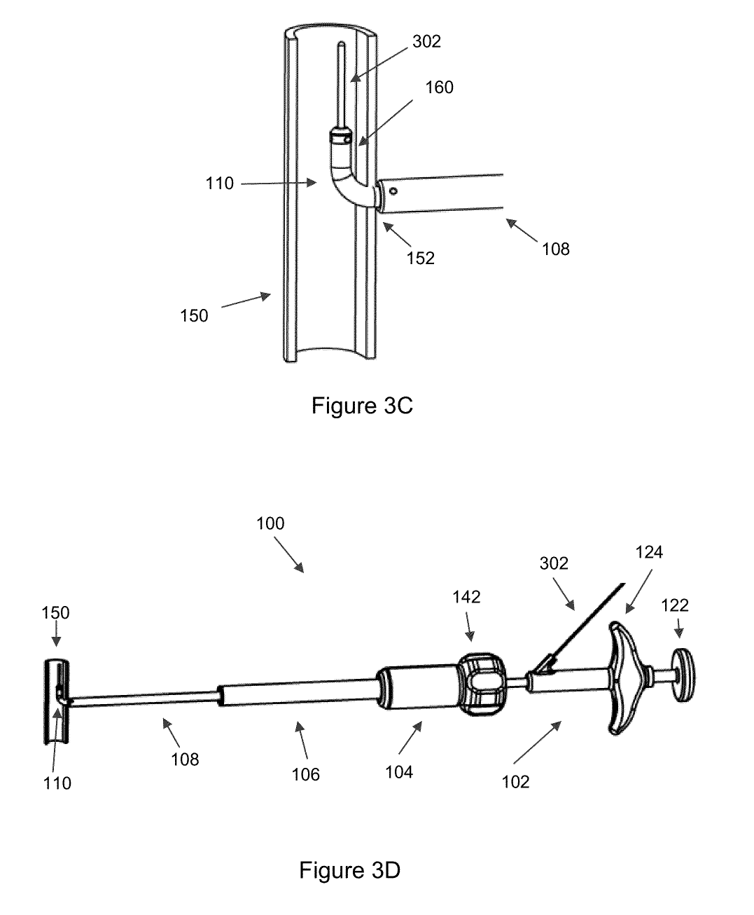

FIG. 3A through FIG. 3D illustrate a guidewire 302 is used to assist with placing the application shaft member 110 through the opening 152 of the lumen 150 to engage the stapling device 100 with the lumen 150 for attaching a tubular structure 902 (see FIG. 9A and FIG. 9B) to the lumen 150. Although a guidewire may be used to facilitate engagement of the stapling device 100 with a lumen 100, it may not be required. Since the stapling device 100 is a substantially elongated and tapered, the application shaft member 110 may be sufficiently small, controllable, and/or maneuverable (for example, in some embodiments or configurations, the application shaft member 110 may be sufficiently stiff, flexible, or steerable) that may not require the assistance of a guidewire to locate and pass through the opening 152 to engage with the lumen 150. Continuing with this example, a guidewire 302 is inserted through the port 126 of the stapling device 100, as illustrated in FIG. 3A. The guidewire 302 is pushed or threaded through a pathway or passage of the port 126 all the way through the distal portion or tip of the application shaft member 110, as illustrated in FIG. 3B. The guidewire is then maneuvered into and through the open 152 of the lumen 150. The application shaft member 110 is pushed along to follow the guidewire 302 into and through the opening 152 into the lumen 150, as illustrated in FIG. 3C and FIG. 3D. As can be appropriated, the application shaft member 110 may be maneuvered into and through the opening 152 without the assistance or aid of a guidewire 302.

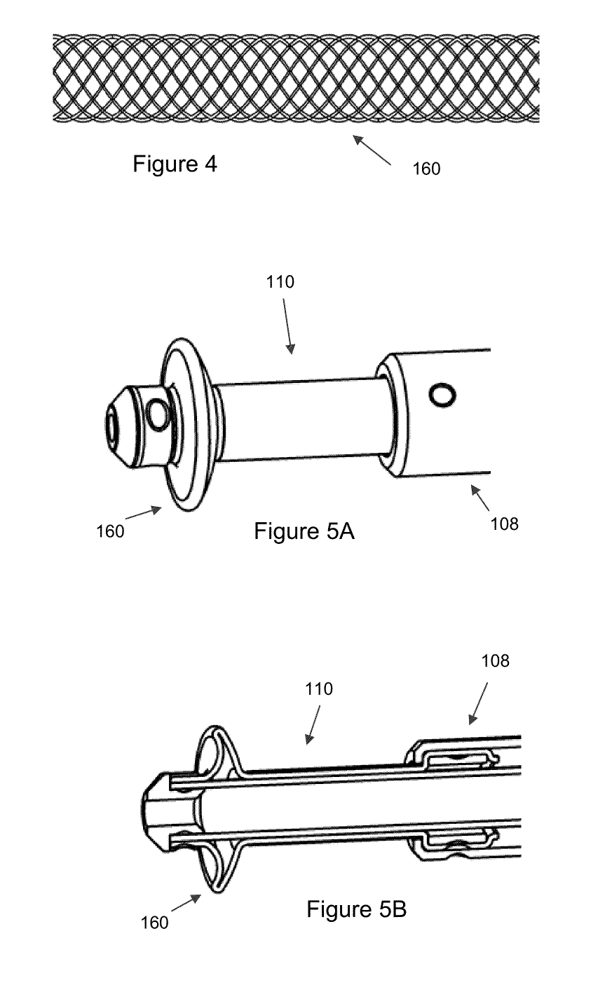

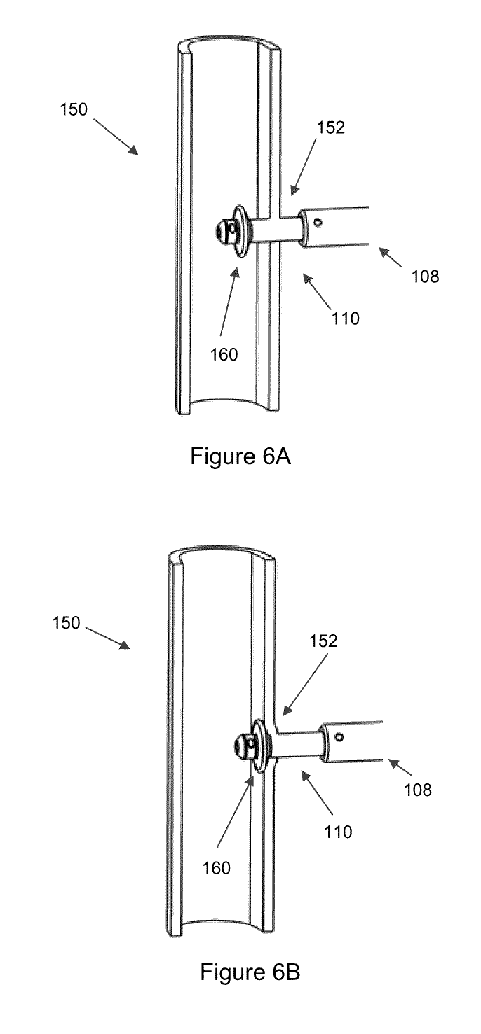

FIG. 4 illustrates that the expandable and contractible support member 160 may be constructed of a mesh-like material or any other suitable material or construction to facilitate expandability and contractibility of the component. FIG. 5A and FIG. 5B illustrate that when the application shaft member 110 is contracted, pulled, or tensioned (e.g., pulling or releasing (may be spring loaded) the control plunder handle 122 from the “pushed” position to an extended or “pulled” position), the expandable and contractible support member 160 is placed into an expanded configuration, such as a substantially disc-like configuration. In the expanded configuration, the support member 160 can support the engagement of the stapling device 100 with the lumen 150 as well as support the application of staples to attach a tubular structure (e.g., a harvested vessel, a prosthesis, etc.) to the lumen 150 (e.g., a blood vessel, a segment of an intestine, etc.). FIG. 6A and FIG. 6B illustrate the expanded support member 160 being used to properly and securely engage the stapling device 100 with the lumen 150.

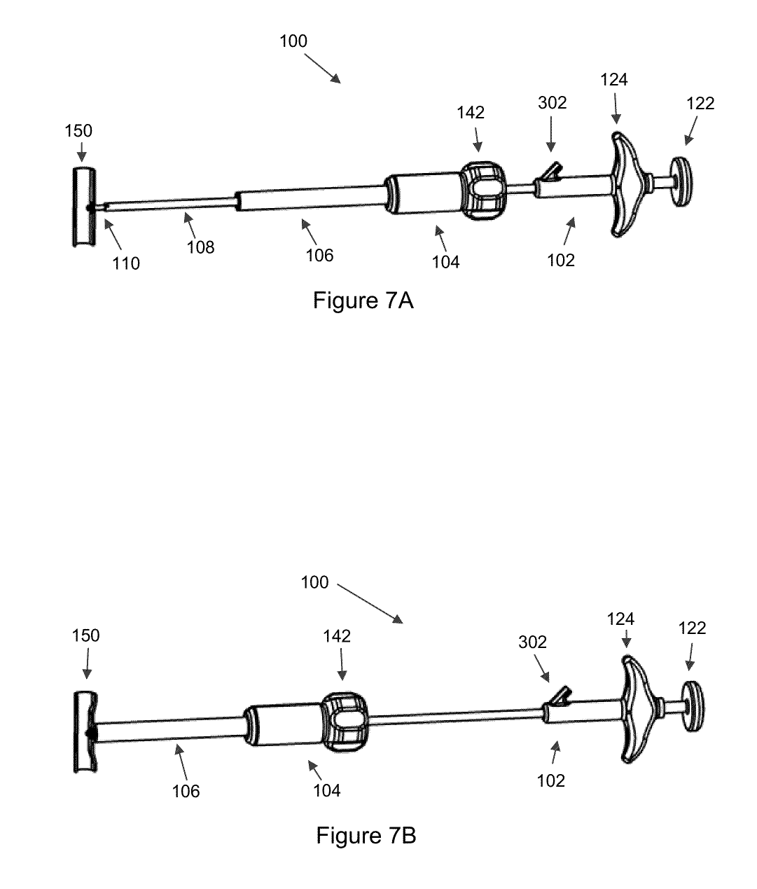

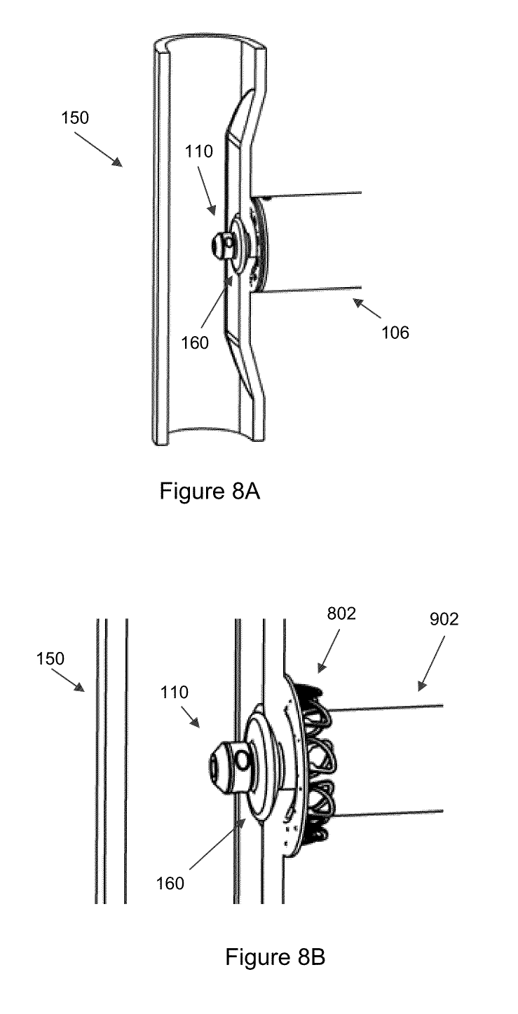

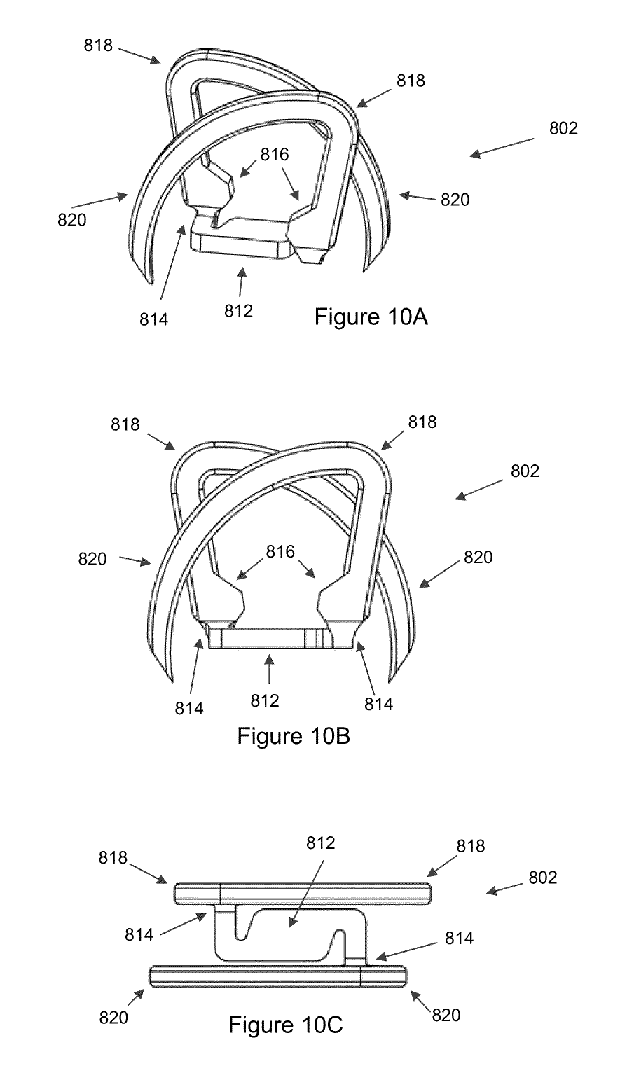

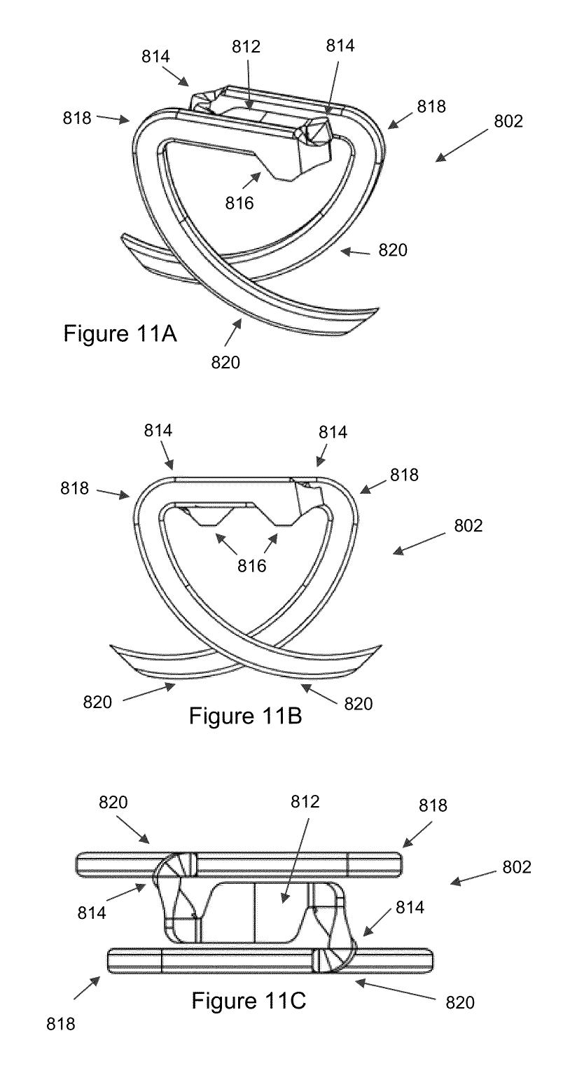

FIG. 7A and FIG. 7B illustrate the start of staple deployment for the attachment of a tubular structure inside of the stapling device 100 to the lumen 150. Picking up from the illustration of FIG. 6B, FIG. 7A illustrates the stapling device 100 has engaged with the lumen 150. Refer to FIG. 8A for a close-up view of this process. FIG. 7B illustrates that the deployment shaft assembly 106 is advanced forward to engage with the lumen to attach a tubular structure 902 (see example illustrated in FIG. 9A and FIG. 9B) with specially configured staples 802. Refer to FIG. 8B for a close-up view of this process. Details of the specially configured staples 802 are illustrated in FIG. 10A through FIG. 10C and FIG. 11A through FIG. 11C.

FIG. 10A illustrates an isometric or perspective view of a specially configured staple/clip of the surgical stapling/clip applying device in its undeployed configuration, in accordance with features, aspects, or embodiments of the present invention. FIG. 10B illustrates a side view of a specially configured staple/clip of the surgical stapling/clip applying device in its undeployed configuration, in accordance with features, aspects, or embodiments of the present invention. FIG. 10C illustrates a top view of a specially configured staple/clip of the surgical stapling/clip applying device in its undeployed configuration, in accordance with features, aspects, or embodiments of the present invention. FIG. 11A illustrates an isometric or perspective view of a specially configured staple/clip of the surgical stapling/clip applying device in its deployed configuration, in accordance with features, aspects, or embodiments of the present invention. FIG. 11B illustrates a side view of a specially configured staple/clip of the surgical stapling/clip applying device in its deployed configuration, in accordance with features, aspects, or embodiments of the present invention. FIG. 11C illustrates a top view of a specially configured staple/clip of the surgical stapling/clip applying device in its deployed configuration, in accordance with features, aspects, or embodiments of the present invention. As illustrated in these figures, the specially configured staples 802 include a base element 812, two hinge elements 814, two stabilizing elements or two first tine elements 816, two curved features or two elbow elements 818, and two legs or two second tine elements 820. The deployment, application, and features of the specially configured staples 802 will be discussed in greater detail.

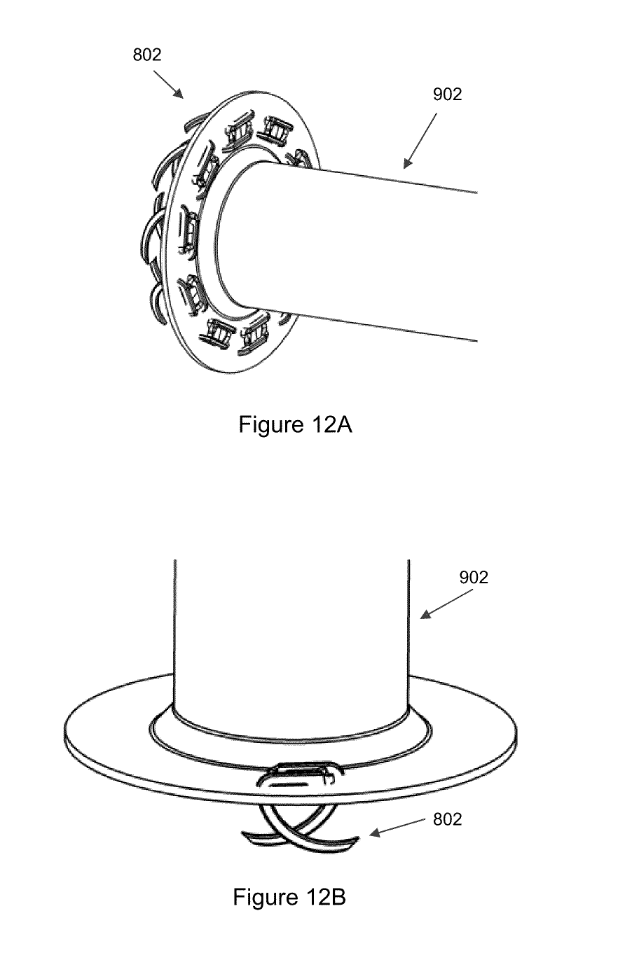

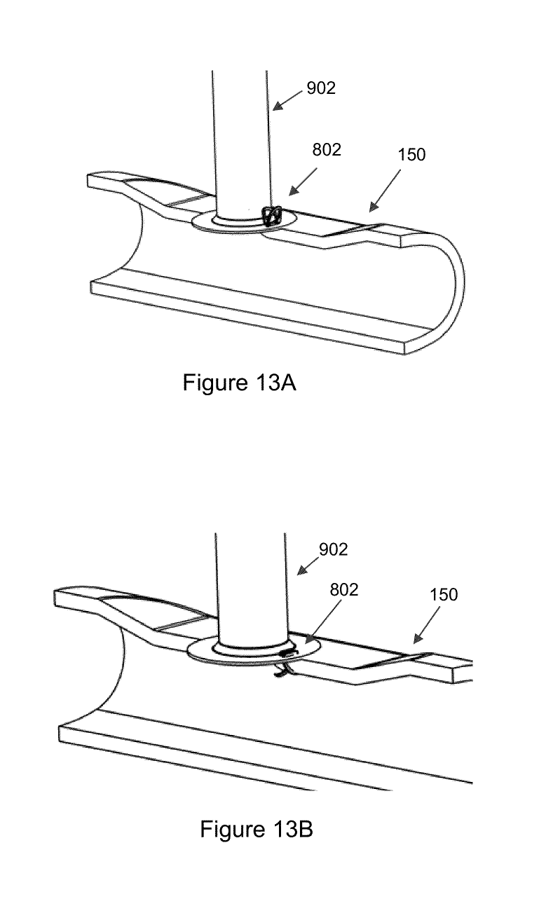

For now, the interactions of the staples 802 and the tubular structure 902 for attachment with the lumen 150 will be illustrated. For sake of clarity, FIG. 12A illustrates the deployed specially configured staples 802 on the tubular structure (without illustrating the lumen 150 being attached with the tubular structure 902). For greater clarity and simplicity, FIG. 12B illustrates one deployed specially configured staple 802 on the tubular structure (without illustrating the lumen 150 being attached with the tubular structure 902). FIG. 13A illustrates a specially configured staple 802 and the tubular structure 902 positioned on the lumen 150 prior to staple deployment. FIG. 13B illustrates the specially configured staple 802 having been deployed to attach or fasten the tubular structure 902 onto the lumen 150. In some applications, the size of the specially configured staples 802 may be tailored specifically to the thickness of the lumen 150, such that there may be a customized fit to the deployed staples 802 with the thicknesses of the tubular structure 902 and the thickness of the lumen 150. In other applications, the specially configured staples 802 may not be specifically sized to the thickness of the tubular structure 902 and the thickness of the lumen 150. As such, in some applications the legs or second tines 820 of the staples 802 may be imbedded into the tissue of the lumen 150 after deployment of the staples 802. In some other applications, the legs or second tines of the staples 802 may “hang-out” with some amount of clearance or space with or between the tissue of the lumen 150 after deployment of the staples 802. In whichever scenario, the staples 802 will securely attach or fasten the tubular structure 902 to the lumen 150. As illustrated in FIG. 11A and FIG. 11B, the legs or second tines 820 of the deployed specially configured staple 802 substantially overlap or cross to provide greater strength of security for attaching or fastening the tubular structure 902 to the lumen 150.

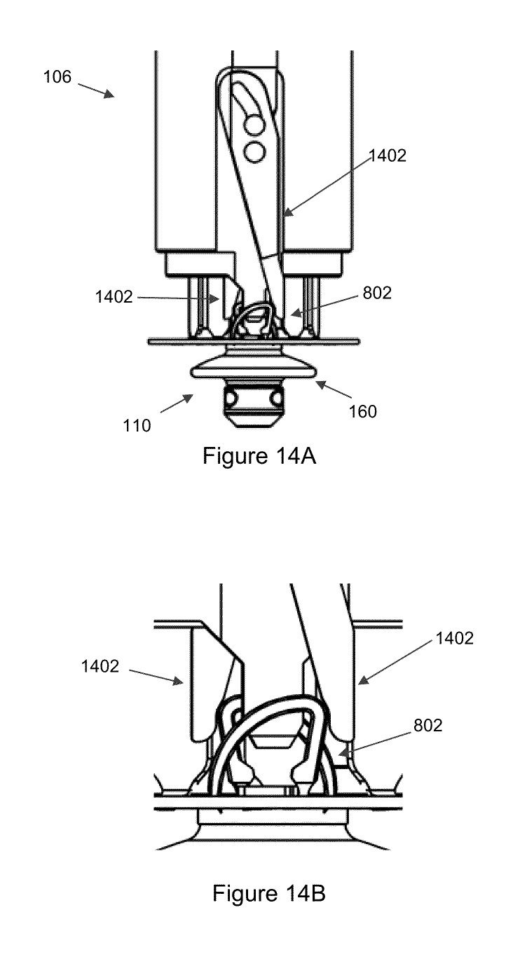

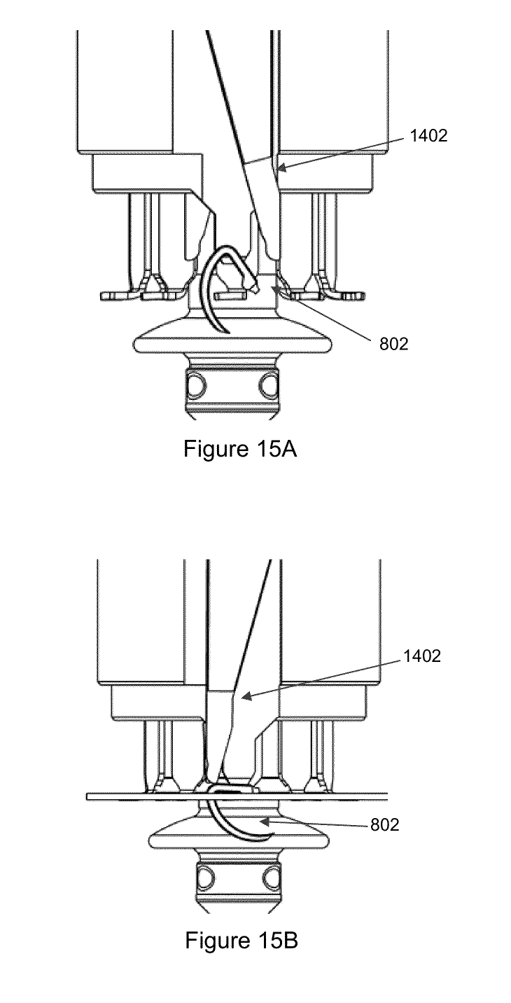

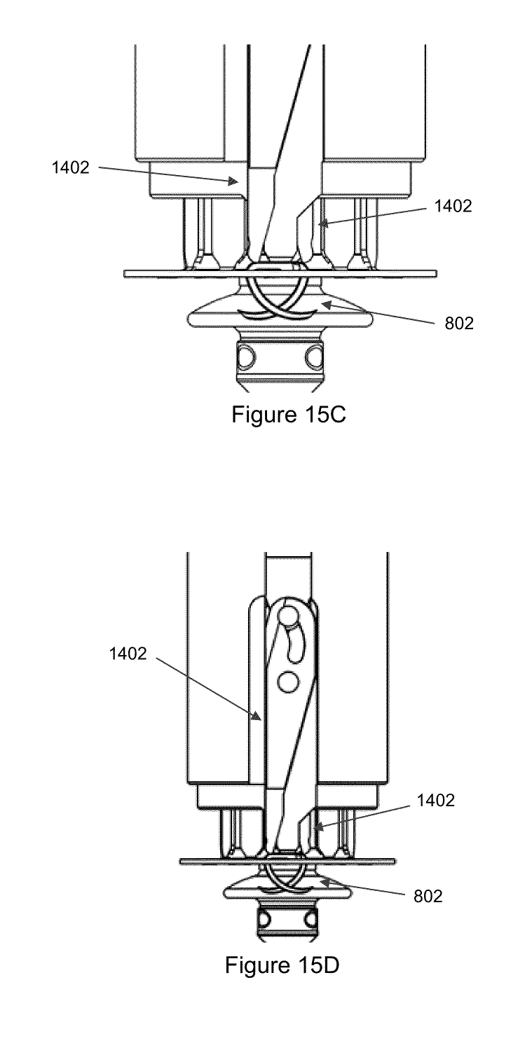

Staple deployment will be illustrated and discussed. FIG. 14A and FIG. 14B illustrate the specially configured staple 802 and the staple deployment mechanisms of the deployment assembly 106. For sake of clarity, the outer shaft member of the deployment assembly 106 is removed to illustrate some of the inner components of the deployment assembly 106. For sake of simplicity, only one specially configured staple 802 is illustrated. As illustrated in FIG. 14A and FIG. 14B, wedge members 1402 are activated to deploy the specially configured staple 802. Wedge members 1402 act in pairs in a scissor-like operation to deploy staple 802. For greater clarity and simplicity, FIG. 15A and FIG. 15B illustrate the movement of one of the wedge members 1402 to urge or push one of the legs or tines for the deployment of the staple 802. As can be appreciated from FIG. 15A and FIG. 15B, as the wedge member 1402 is activated (in this example, the wedge 1402 is moved from right to left) it urges or pushes the staple 802 near its curved or elbow element 818 to a substantially semi-circular or arcuate motion about its hinge element 814. As movement or motion of the leg or tine 820 is initiated, the stabilizing element or first tine engages with the tubular structure 902 and then the tissue of the lumen 150 to stabilize and/or anchor the tine structure as the second tine element engages or pierces the tubular structure 902 and then the tissue of the lumen 150. In effect, the first tine helps to ensure stable movement or swing of the semi-circular or arcuate motion of the entire leg or tine element of the staple 802. Expressed in another way, the first tine 816 “bites” into or anchors “down-into” the tubular structure 902 and/or the tissue of the lumen 150 to prevent unstable movement and/or twisting of the relative slender length of the leg or tine of the staple 802. FIG. 15C and FIG. 15D illustrate the typical deployment operation as both wedge elements 1402 are operated at substantially the same time to urge or push both legs or tines of the staple 802 for stapling or clipping the tubular structure 902 to the lumen 150. As can be appreciated from FIG. 10A through FIG. 10C and FIGS. 11A through 11C, the hinge elements 814 and the pivot points of the legs or tines of the staple 802 are offset from each other at a distance about the width of the base member 812. In other words, the hinge elements 813 or pivot points are typically non-collinear along the axis of the base member 812. Also, the hinge elements 813 may be positioned or oriented on the opposite sides of the based member 812. Typically, the hinge elements 813 are positioned at or near of ends of the base member 812. Alternatively, the hinge elements 813 can be positioned substantially anywhere along the length of the base member 812. When the staple is deployed, the legs or tines of the staple would not interfere with one another; however, as discussed previously, the legs or tines overlap or cross (as seen from the side view) to provide greater strength of security for attaching or fastening the tubular structure 902 to the lumen 150.

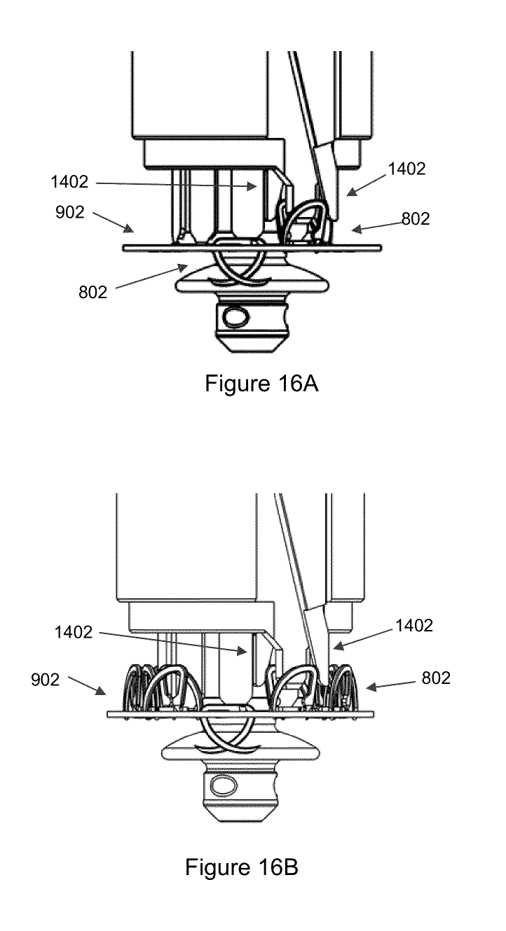

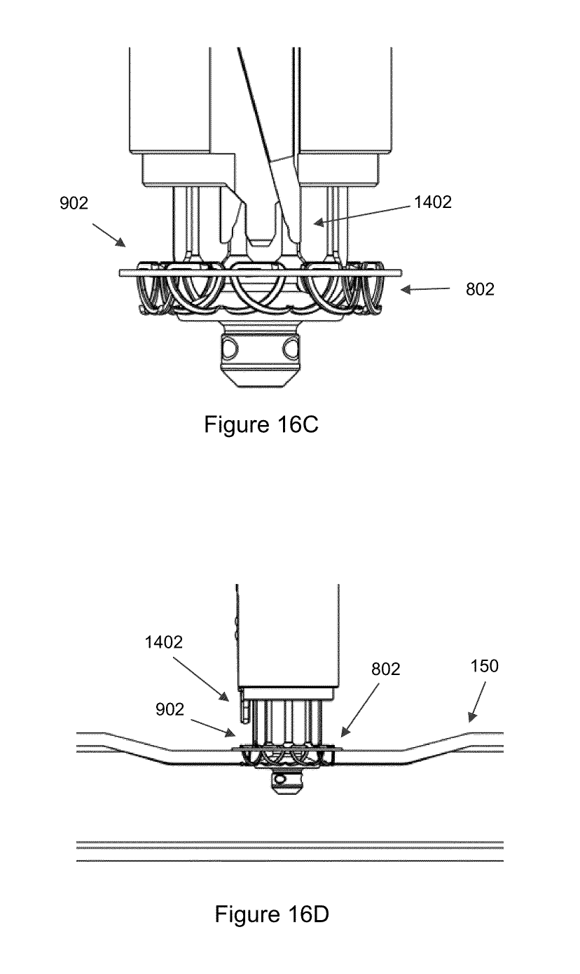

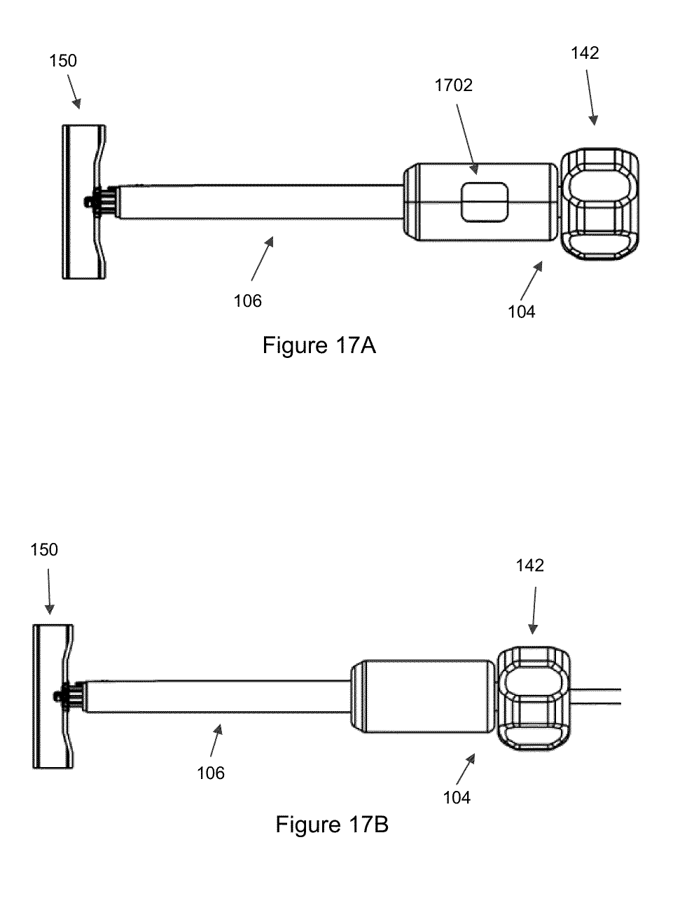

FIG. 16A through FIG. 16C illustrates sequential activation and deployment of the staples 802 by the wedge members 1402, without illustrating the lumen 150 for sake of simplicity. FIG. 16D illustrates that the staples have been deployed to attach the tubular structure 902 to the lumen 150. Sequential activation and deployment of the staples 802 by the wedge members 1402 may be controlled by the deployment control knob member 142 working in concert with the deployment control index member 1702 (see FIG. 17A). For example, activation of the wedge members 1402 may be done by twisting the deployment control knob member 142, and advancing the wedge members 1402 from one staple 802 to the next staple 802 may be done by activating the deployment control index member 1702, as illustrated in FIG. 17A. Optionally, activation of the wedge members 1402 may be done by twisting the deployment control knob member 142 in first direction, while advancing the wedge members 1402 to another staple 802 may be accomplished by twisting the deployment control knob member in a second direction that is opposite to the first direction, as illustrated in FIG. 17B. In such an optional configuration, the deployment control knob control index member 1702 may not be necessary.

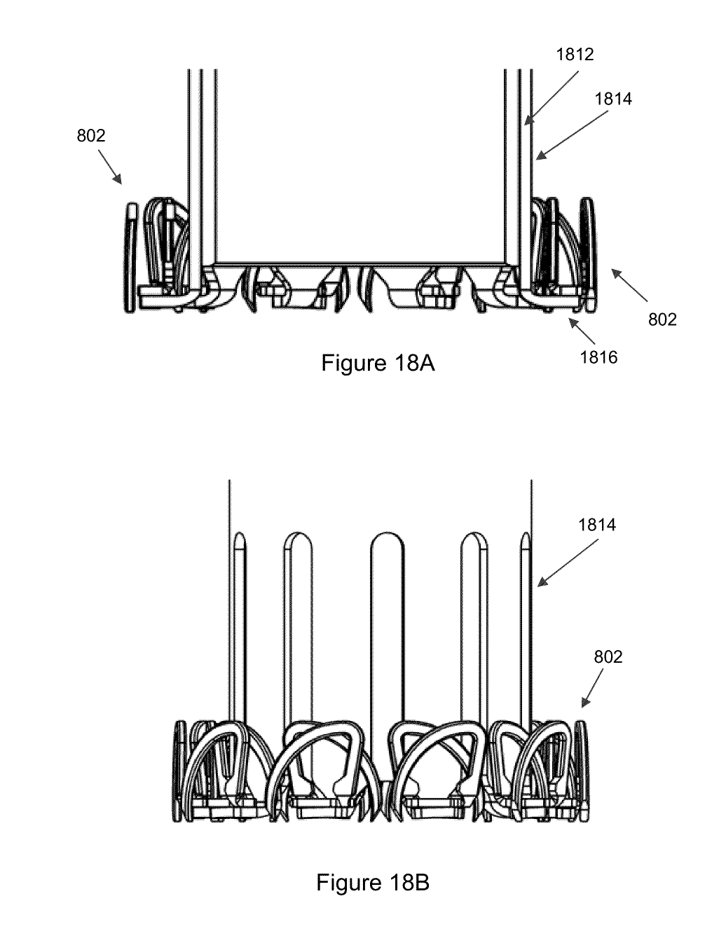

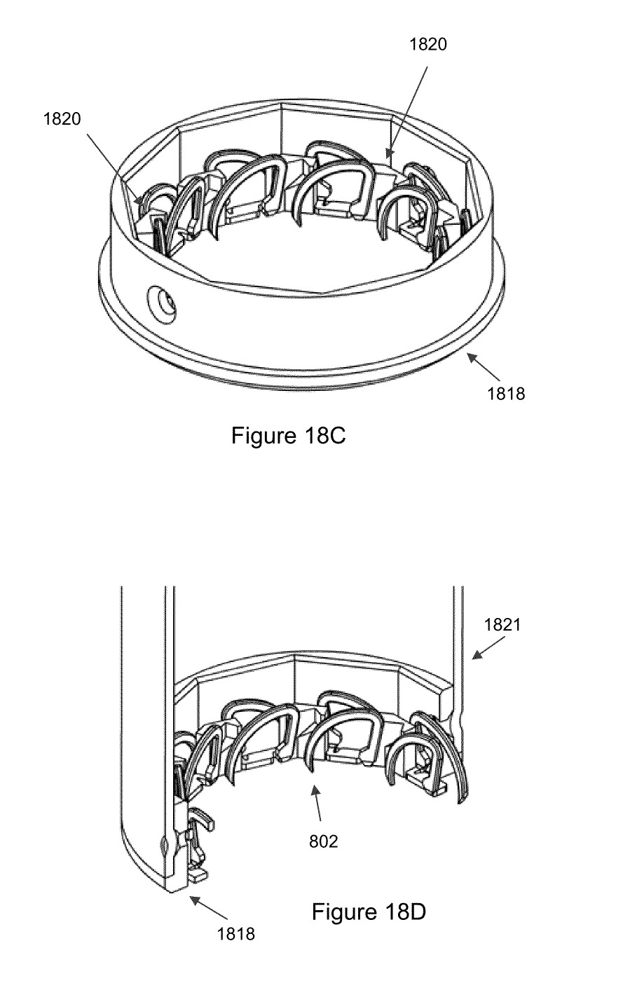

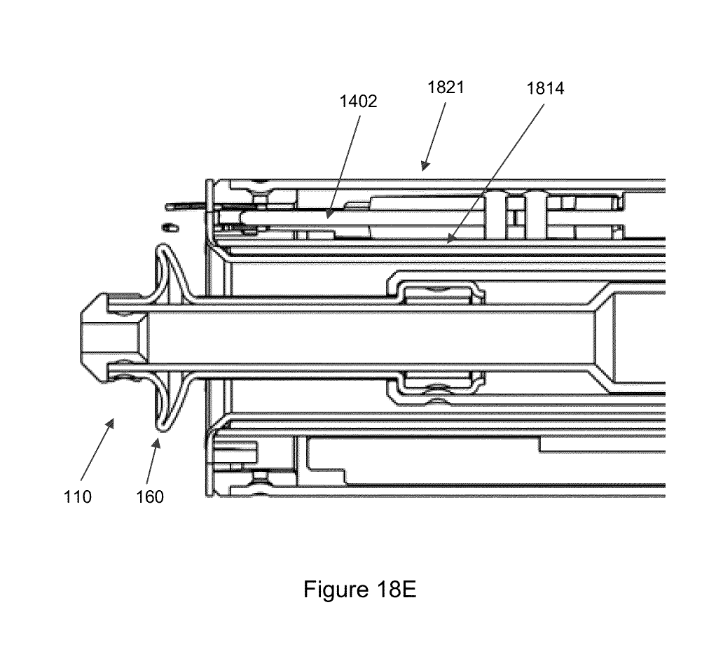

FIG. 18A through FIG. 18E provide further structural details of the deployment shaft member assembly 106. FIG. 18A illustrates a cross-sectional view of the deployment shaft member assembly 106. As illustrated, the deployment shaft member assembly 106 includes an inner support tube member 1812 and a staple or clip support arm member 1814. In some embodiments, the inner support tube member 1812 may act as a structure member of the deployment shaft member assembly 106. Also, in some embodiments, the support arm member 1814 may provide detachable support to the specially configured staples or clips 802. FIG. 18B illustrates a side-view of the staple or clip support arm member 1814 and staples 802 to show the staples 802 being coupled to staple support arm member 1814. Each of the staples 802 may be detachably supported by a support finger member 1816. Once the staples/clips 802 are deployed, they can slide-off the support finger member 1816. In addition, the support finger member 1816 may also be substantially flexible to allow easy decoupling or detachment of the staples/clips 802 once they are deployed. Furthermore, once the staples 802 are deployed, the deployment shaft assembly member 106 may be withdrawn or slide-away from the deployment position; as such, withdrawing the deployment shaft assembly member 106 may also facilitate detachment of the staples 802 from the stapling device 100. In addition to the support arm member 1814, the staples 802 may also be supported or secured by a staple or clip constraint member 1818 configured with staple/clip support elements 1820 to hold or support the staples/clips 802 in their undeployed state. Once the staples/clips 802 are deployed the staple/clip support elements 1820 no longer hold or support the staples/clips 802 in their deployed state. In other words, the staple/clip support elements 1820 are configured or shaped to hold or support the staples/clips 802 when the staples/clips are in their undeployed configuration (see FIG. 10A through FIG. 10C for staples in undeployed configuration and FIG. 11A through FIG. 11C for staples in deployed configuration). As such, the undeployed staples 802 may be secured with only the support arm member 1814 or the constraint member 1818 with the support features 1820 or the combination of the support arm member 1814 and the constraint member 1818. FIG. 18D illustrates a cross-sectional view of the staple/clip outer support tube member 1821 of the stapling device 100 with the staple support element 1820 to provide additional details of the structure and construction of the deployment shaft assembly 106. To provide further perspective of the structures and construction of the stapling device 100, FIG. 18E illustrates a cross-sectional view of the distal portion of the surgical stapling/clip applying device illustrating the various structures and members of the device located at the distal portion of the device.

Multiple features, aspects, and embodiments of the invention have been disclosed and described herein. Many combinations and permutations of the disclosed invention may be useful in anastomosis surgical procedures, and the invention may be configured to support various grafting procedures. One of ordinary skill in the art having the benefit of this disclosure would appreciate that the foregoing illustrated and described features, aspects, and embodiments of the invention may be modified or altered, and it should be understood that the invention generally, as well as the specific features, aspects, and embodiments described herein, are not limited to the particular forms or methods disclosed, but also cover all modifications, equivalents and alternatives. Further, the various features and aspects of the illustrated embodiments may be incorporated into other embodiments, even if not so described herein, as will be apparent to those ordinary skilled in the art having the benefit of this disclosure.

Although particular features, aspects, and embodiments of the present invention have been illustrated and described, it should be understood that the above discussion is not intended to limit the present invention to these features, aspects, and embodiments. It will be obvious to those skilled in the art that various changes and modifications may be made without departing from the spirit and scope of the present invention. Thus, the present invention is intended to cover alternatives, modifications, and equivalents that may fall within the spirit and scope of the following claims and their equivalents.

What is claimed:

1. An apparatus for performing anastomosis procedures, comprising:
an end-effector to engage a lumen for attaching a tubular structure to said lumen to create a connective branch or an anastomosis;
a contractible and expandable member coupled to the end-effector to secure engagement of the end-effector with the lumen,
a pair of wedge members configured to deploy a staple for attaching the tubular structure to the lumen, wherein the pair of wedge members operates in opposing directions to deploy said staple.

2. The apparatus of claim 1, wherein the pair of wedge members operate in a scissor-like motion to deploy said staple.

3. The apparatus of claim 1, wherein the contractible and expandable member provides support to the lumen as the staple is deployed to attach the tubular structure to the lumen.

4. The apparatus of claim 1, wherein the contractible and expandable member is placed in a contracted state when the end-effector is inserted through the lumen for engagement.

5. The apparatus of claim 1, wherein the contractible and expandable member is placed in an expanded state to secure the engagement of the end-effector with the lumen.

6. The apparatus of claim 1, wherein the end-effector can be operated telescopically by extending from a first length to a second length, retracting from the second length to the first length, or varying at a length between the first length and the second length.

7. The apparatus of claim 1, wherein the end-effector is substantially rigid, substantially flexible, or substantially steerable.

8. The apparatus of claim 1, wherein the wedge members deploy the staple by urging leg members of the staple in respective arcuate motions for staple deployment.

9. A staple for performing anastomosis procedures, comprising:
a base member with a hinge element near each of two ends of said base member, wherein the hinges are oriented at laterally opposite sides of the base member;
a leg member extends from each of the hinge elements, wherein each leg member includes a first tine and a second tine.

10. The staple of claim 9, wherein each leg member pivots or rotates respectively about the hinge member in an arcuate motion when the staple is deployed.

11. The staple of claim 9, wherein, in a deployed state, the leg member of one end of the base member overlaps or crosses with another leg member of another end of the base member.

12. The staple of claim 9, wherein the hinge elements are non-collinear with an axis of the base member.

13. The staple of claim 9, wherein each of the hinge elements becomes twisted after the staple is deployed.

14. The staple of claim 9, wherein each of the hinge elements becomes twisted after the staple is deployed due to respective rotation of the leg members.
